(19) United States
(12) Patent Application Publication
(10) Pub. No.: US 2019/0343444 A1
(43) Pub. Date: Nov. 14, 2019

(54) URINE INFORMATION PROCESSING DEVICE, URINE INFORMATION PROCESSING SYSTEM, AND STORAGE MEDIUM

(71) Applicant: SHARP KABUSHIKI KAISHA, Sakai City, Osaka (JP)

(72) Inventors: YUHICHI YABUKI, Sakai City (JP); HIROKAZU ISHIKAWA, Sakai City (JP)

(21) Appl. No.: 16/461,506

(22) PCT Filed: Aug. 31, 2017
(86) PCT No.: PCT/JP2017/031457

§ 371 Date: May 16, 2019

(30) Foreign Application Priority Data
Nov. 18, 2016 (JP) 2016-225459

(51) Int. Cl. A61B 5/20; A61B 10/00; A61B 5/00

(52) U.S. Cl. CPC A61B 5/207; A61B 10/007; A61B 8/5223; A61B 5/202; A61B 5/6891

(57) ABSTRACT

The obtain information on the urination amount of a measurement target arid determine the health status of the measurement target on the basis of the information obtained, a CPU (1) of a urination amount measurement device (100) compares the urination amount detected by a sensor (31) with a threshold value corresponding to the body weight of the measurement target to determine the health status of the measurement target, and an output section (9) outputs the result of the determination by the CPU (1).

### TECHNICAL FIELD

The present invention relates to a urine information processing device for processing information on urination. The present invention relates to a urine information processing device and the like for determining the health status of a measurement target person on the basis of information on urination by the measurement target person.

### BACKGROUND ART

Information on urination, e.g. urination amount, urination time, and the number of times of urination, includes various pieces of information on a sign of disease of a patient or an animal (measurement target). There has been a urine information processing device for measuring information related to urination and outputting data obtained as a result of the measurement (see, for example, Patent Literature 1 and Patent Literature 2). Data outputted by conventional device urination information processing is based on the following assumption. That is, a medical specialist or the like judges the presence or absence of a sign of disease from the data on the basis of their own knowledge.

### CITATION LIST

### Patent Literature

### Patent Literature 1

Japanese Patent Application Publication, Tokukai, No. 2002-186601 A (Publication Date: Jul. 2, 2002)

### Patent Literature 2

Japanese Patent Application Publication, Tokukai, No. 2008-206592 A (Publication Date: Sep. 11, 2008)

### SUMMARY OF INVENTION

### Technical Problem

A normal user who is not a medical specialist does not have knowledge like a medical specialist does. Therefore, it is difficult for the user to determine the health status of the user or a pet, etc. on the basis of data of a conventional urine information processing device.

The Applicant investigated the urination amount per time or per day of a measurement target, etc. As a result, the following was found. That is, when a measurement target is suffering from a particular disease such as urinary disease or kidney disease, the average urination amount of the measurement target tends to differ significantly from the average urination amount of a healthy measurement target (see the data shown in FIG. 12). As a conclusion of the above investigation, the Applicant arrived at the following idea. That is, the health status of a measurement target can be easily determined on the basis of the urination amount.

The present invention has been made in view of the above issue. It is an object of the present invention to provide a urine information processing device configured to obtain information on the urination amount of a measurement target and determine the health status of the measurement target on the basis of the information obtained.

### Solution to Problem

In order to attain the above object, a urine information processing device in accordance with an aspect of the present invention is a urine information processing device including: a urination amount measurement section configured to measure an amount of urination by a measurement target that uses a toilet tool; a comparison section configured to compare (i) the amount of the urination by the measurement target, the amount having been measured by the urination amount measurement section, with (ii) a predetermined value corresponding to a body weight of the measurement target; a health status determination section configured to determine a health status of the measurement target on a basis of a result of the comparison by the comparison section; and a determination result presentation section configured to present a result of the determination by the health status determination section.

In order to attain the above object, a urine information processing device in accordance with an aspect of the present invention is a urine information processing device including: a urination information measurement section configured to (i) sense a measurement target that uses a toilet tool and (measure either the number of times of urination by the measurement target over a predetermined time period or a time period necessary for the measurement target to urinate; a comparison section configured to compare either the number of times of the urination by the measurement target, the number having been measured by the urination information measurement section, with a first predetermined value for the number of times of urination or the time period necessary for the measurement target to urinate, the time period having been measured by the urination information measurement section, with a first predetermined value for a time period necessary for urination; a health status determination section configured to determine a health status of the measurement target on a basis of a result of the comparison by the comparison section, and a determination result presentation section configured to present a result of the determination by the health status determination section.

### Advantageous Effects of Invention

An aspect of the present invention enables information on the urination amount of a measurement target to be obtained and the health status of the measurement target to be determined on the basis of the information obtained.

### BRIEF DESCRIPTION OF DRAWINGS

FIG. 1 is a block diagram illustrating the configuration of functions carried out by a CPU included in a urination amount measurement device according to Embodiment 1.

FIG. 2 is a block diagram illustrating the configuration of a urination amount measurement device according to Embodiment 1.

FIG. 3 is a block diagram illustrating the configuration of a sensor according to Embodiment 1.

FIG. 4 is a diagram illustrating a toilet room in which a toilet tool used by a measurement target person is present, and is a diagram schematically illustrating a urine information processing system according to Embodiment 1.

FIG. 5 is a cross-sectional view of a toilet tool installed in the toilet room shown in FIG. 4.

FIG. 6 is a flowchart illustrating the flow of a health status determining process carried out by a urination amount measurement device according to Embodiment 1.

FIG. 7 is a flowchart illustrating the flow of a urination amount information storing process carried out by a urination amount measurement device according to Embodiment 1.

FIG. 8 is a diagram schematically illustrating the configuration of a database stored by a storage section included in a urination amount measurement device according to Embodiment 1.

FIG. 9 is a flowchart illustrating the flow of a urination amount information storing process according to Embodiment 2.

FIG. 10 is a diagram schematically illustrating the configuration of a urination amount measurement device according to Embodiment 3.

FIG. 11 is a diagram schematically illustrating a urine information processing system according to Embodiment 6.

FIG. 12 is exemplary data illustrating the correlation between the urination amount and particular diseases.

### DESCRIPTION OF EMBODIMENTS

The following description discusses embodiments with reference to drawings.

### Embodiment 1

### Systems Overview

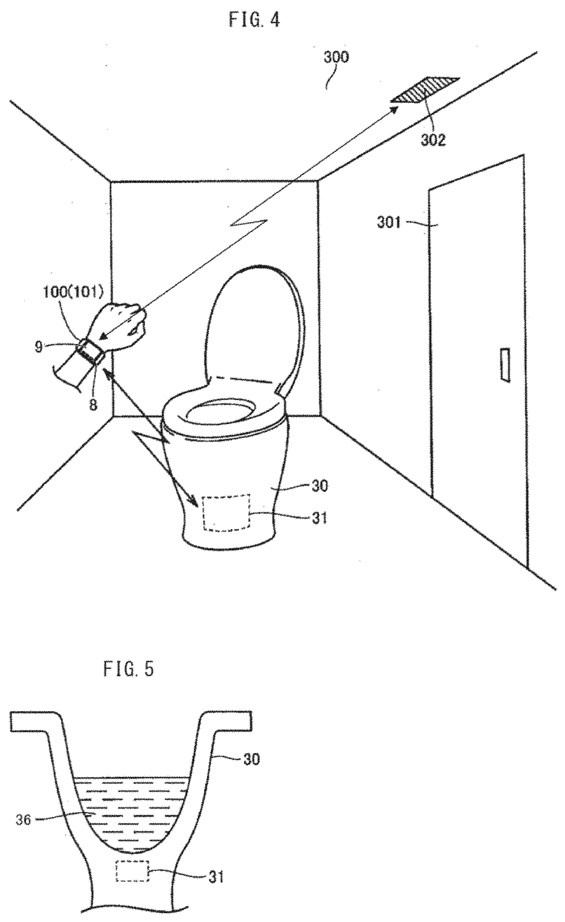

FIG. 4 is a diagram schematically illustrating a toilet room 300 according to Embodiment 1. FIG. 5 is a cross-sectional view of a toilet tool 30 installed in the toilet room 300.

As shown in FIG. 4, the toilet room 300 includes a door 301, a human sensor 302 (measurement target detection section), and a toilet tool 30. The urine information processing system according to the present embodiment includes a urination amount measurement device 100, a sensor 31 (urination detection section), and a human sensor 302. As shown in FIGS. 4 and 5, the sensor 31 is installed at a bottom portion of the toilet tool 30. As shown in FIG. 5, the toilet tool 30 also holds collected water 36. A measurement target person's urine falls into the collected water 36. The sensor 31 is located below the collected water 36.

A water level sensor section 34 included in the sensor 31 detects that urine has fallen into the collected water 36. The water level sensor section 34 stores, in a memory 33 included in the sensor 31, data (for example, data on a pressure or weight described later) including information related to the water level. A CPU 32 included in the sensor 31 uses data stored in the memory 33 to generate data including information related to the urination amount to output the generated data to the urination amount measurement device 100 (urine information processing device). A communication section 35 included in the sensor 31 transmits, to the urination amount measurement device 100, data including information related to the urination amount. The human sensor 302 detects, with use of infra-red rays or the like, a measurement target person who opens the door 301 and enters the toilet room 300 or opens the door 301 and exits the toilet room 300. In a case where the human sensor 302 has detected a measurement target person who enters the toilet room 300 or exits the toilet room 300, the human sensor 302 sends a predetermined detection signal to the urination amount measurement device 100.

### Configuration of Urination Amount Measurement Device 100

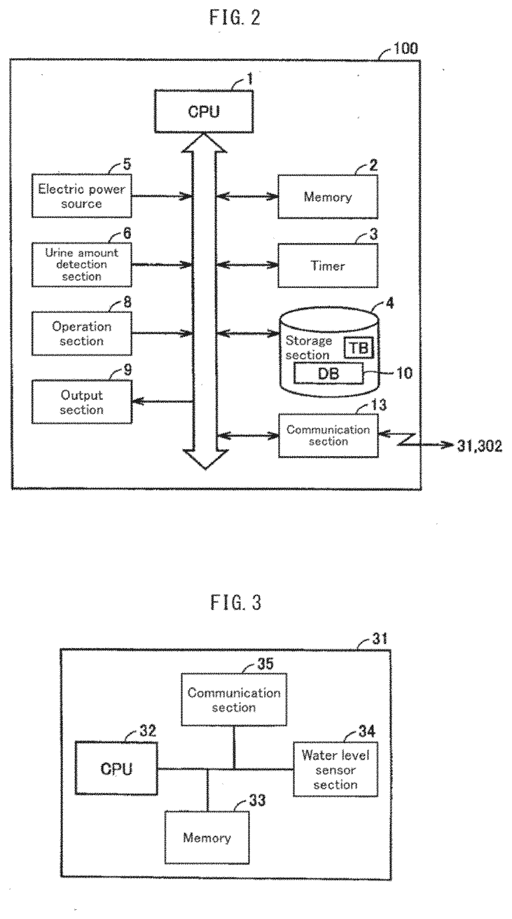

FIG. 2 is a diagram schematically illustrating the configuration of a urination amount measurement device 100 according to the present embodiment. The urination amount measurement device 100 may be, for example, an information processing terminal worn by a measurement target person (e.g., a smart watch), as shown in FIG. 4. Alternatively, the urination amount measurement device 100 may be a small information processing terminal (e.g., a smartphone) that can be carried by the measurement target person. The urination amount measurement device 100 receives data including information related to the urination amount from the sensor 31 (see FIGS. 4 and 5) described above. The urination amount measurement device 100 measures the urination amount of the measurement target person and determines the health status of the measurement target person on the basis of the data received from the sensor 31. The urination amount measurement device 100 also outputs the result of determining the health status of the measurement target person to the output section 9. In addition, the urination amount measurement device 100 may output the result of determining the health status of the measurement target person to an external device (for example, a monitor fixed to a wall surface of the toilet room 300 or the like).

The measurement target person checks information presented and displayed in the output section 9 or the external device above. This facilitates checking whether the user's health status is good or whether the user's health status may be becoming bad. The measurement target of the present embodiment is a human being. The measurement target may be an animal such as a dog or a cat. In this case, the owner or the like of the animal as the measurement target checks the presented health status of the measurement target.

As shown in FIG. 2, the urination amount measurement device 100 includes a central processing unit (CPU) 1, a memory 2, a timer 3, a storage section 4, an electric power source 5, a urine amount detection section 6, an operation section 8, an output section 9, and a communication section 13. The operation section 8 includes, for example, at least one of a button, a switch, and a touch pad. The communication section 13 makes a radio communication with the sensor 31 and the human sensor 302 (see FIG. 4) described above. The memory 2 includes a read only memory (ROM) and a random access memory (RAM). The timer 3 is used by the CPU 1 to measure urination time, as will be described later.

The storage section 4 stores a database 10 including data on the urination amount received from the sensor 31. The storage section 4 may be included in the urination amount measurement device 100. The storage section 4 may be detachable from the urination amount measurement device 100. The storage section 4 further stores a table. The table shows the correspondence between the body weight and a threshold value. The threshold value may be set in any manner. For example, it may be set as follows. That is, a medical doctor has knowledge about the correlation between a particular disease and the urination amount. The medical doctor may be aware of the average urination amount of healthy measurement target persons, e.g., 15 ml/kg. This is stored as a threshold value in the table. Using a threshold value to determine whether or not a measurement target is suffering from a particular disease (i.e., whether or not a measurement target is likely to be suffering from a particular disease) makes it possible to determine whether or not the measurement target is suffering from the disease. For example, using a threshold value calculated from the urine amount with respect to the body weight of a measurement target suffering from urinary calculus and the urine amount with respect to the body weight of a measurement target not suffering from urinary calculus makes it possible to even determine whether the measurement target is suffering from urinary calculus. Using a similar threshold value allows a measurement target to determine whether or not the measurement target is suffering from renal failure as well.

Alternatively, the threshold value may be set on the basis of the body weight of a measurement target person and the average value of urination amounts previously detected by the sensor 31. The reason for this is that in a case where the urine amount has been significantly reduced or in a case where the urine amount has been significantly increased, or in a case where such a trend continues, there may be a problem with the health status of the measurement target person. For example, assuming that the body weight of the measurement target person is A {=A1, A2, . . . } and the average value of urination amounts of the measurement target person is a {=a1, a2, . . . }, the table indicates the corresponding relation of {A1/a1, A2/a2, . . . }. In this case, the threshold value for a case where the body weight of a measurement target person is A1 may be set to have, for example, an upper limit value of a1+α and/or a lower limit value of a1−β. In this example, in a case where the urination amount of a measurement target person exceeds the upper limit value a1+α, or in a case where the urination amount of a measurement target person falls below the lower limit value a1−β, the health status of the measurement target person may be becoming bad. According to knowledge of the Applicant, it is desirable that α is, for example, a1×10%, and β is, for example, a1×30%. Thus, the lower limit value a1−β may be more deviated from a1 than the upper limit value a1+α. On the other hand, in a case where the urination amount of a measurement target person is greater than the lower limit value of a−β and is less than the upper limit value a+α, the health status of the measurement target person is good. The following description uses the term “normal” to refer to when the urination amount is greater than the lower limit value a−β and less than the upper limit value a+α. The following description uses the term “abnormal” to refer to both when the urination amount is not less than the upper limit value a+α and when the urination amount is not more than the lower limit value a−β. That is, the following description refers to the fact that the urination amount is deviated from a normal value by not less than a certain value (α, β) as being “abnormal”.

The output section 9 includes a display section (display). The output section 9 may further include an audio output section such as a loudspeaker. The touch pad included in the operation section 8 and the display section included in the output section 9 may be integrated in the form of a touch panel.

The communication section 13 communicates with the communication section 35 included in the sensor 31 and receives data including information related to the urination amount. The communication section 13 outputs the data received from the sensor 31 to the urine amount detection section 6. The urine amount detection section 6 arranges, in time series, the data received by the communication section 13 and outputs the arranged data to the CPU 1. In a case where the sensor 31 has a weight sensor instead of the water level sensor section 34, the urine amount detection section 6 calculates the urination amount on the basis of data on the weight of the collected water 36.

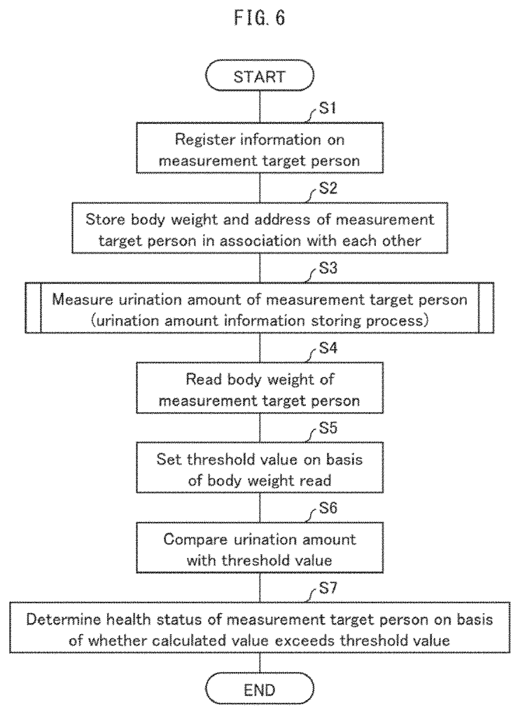

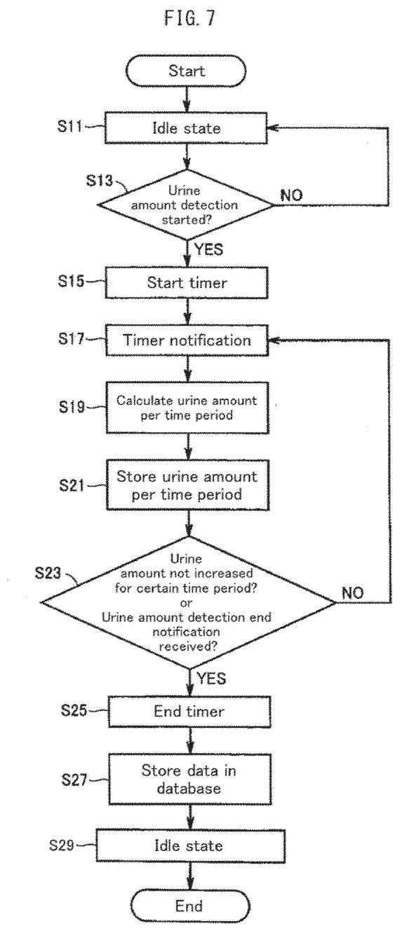

In a case where the CPU 1 has received from the sensor 31 data including information related to the urination amount, the CPU 1 executes a program stored in the memory 2 or the storage section 4. This allows a health status determining process (see FIG. 6) and a urination amount information storing process (see FIG. 7) to be carried out. The storage medium on which to store the program may be “a non-transitory tangible medium” such as a tape, a disk, a card, a semiconductor memory, and a programmable logic circuit. Further, the program may be supplied to or made available to the urination amount measurement device 100 via any transmission medium (such as a communication network and a broadcast wave) which enables transmission of the program. Note that one aspect of the present invention can also be implemented by the program in the form of a computer data signal embedded in a carrier wave which is embodied by electronic transmission.

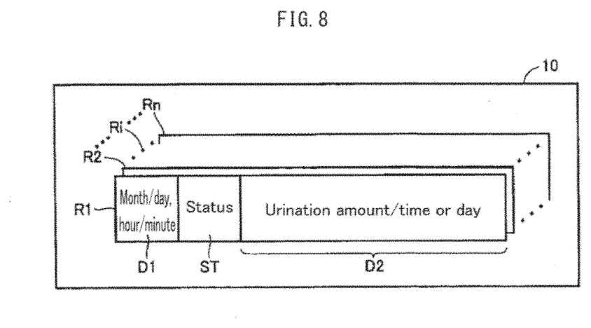

During the urination amount information storing process, the CPU 1 generates record data of the urination amount (see FIG. 8) and stores the generated record data in the DB 10 of the storage section 4. In addition, during the health status determining process, the CPU 1 determines the health status of a measurement target person on the basis of the urination amount per time or per day. The CPU outputs the result of the determination to the output section 9 and/or the external device.

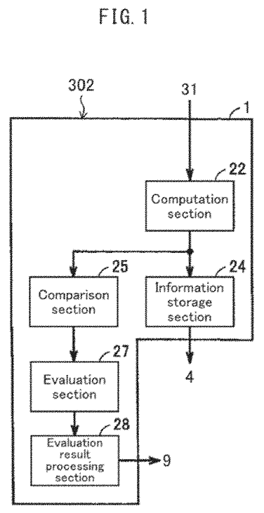

FIG. 1 is a block diagram illustrating functions to be carried out by the CPU 1. As shown in FIG. 1, the CPU 1 includes a computation section 22 (urination amount measurement section, urination information measurement section), an information storage section 24, a comparison section 25, an evaluation section 27 (health status determination section), and an evaluation result processing section 28 (determination result presentation section). The processes carried out by the individual sections included in the CPU 1 will be described in the description below of the health status determining process and the urination amount information storing process.

### Configuration of Sensor 31

FIG. 3 is a block diagram illustrating the configuration of the sensor 31 installed in the toilet tool 30. As shown in FIG. 3, the sensor 31 includes a CPU 32, a memory 33, a water level sensor section 34, and a communication section 35. The water level sensor section 34 may be, for example, of a pressure-type or an ultrasonic type. The water level sensor section 34 detects the water level of the collected water 36 shown in FIG. 5. The communication section 35 communicates with the communication section 13 included in the urination amount measurement device 100, and transmits data including information related to the urination amount to the urination amount measurement device 100. Specifically, the sensor 31 transmits information on the water level (in milliliters) detected by the water level sensor section 34 to the urination amount measurement device 100 as information on the urination amount. The sensor 31 may include a weight sensor instead of the water level sensor section 34. In this case, the sensor 31 transmits information on the weight of the collected water 36 detected by the weight sensor to the urination amount measurement device 100 as information on the urination amount.

### Flow of Health Status Determining Process

FIG. 6 is a flowchart illustrating the flow of a health status determining process carried out by the CPU 1 included in the urination amount measurement device 100. The memory 2 stores a program corresponding to the flowchart. The CPU 1 reads the program from the memory 2 and executes the program.

As shown in FIG. 6, the CPU 1 first registers a measurement target person as a user of service (S1). The CPU 1 stores in the storage section 4 data that associates (i) information on the body weight inputted by the measurement target person with (ii) address information for specifying the measurement target person. (S2).

Thereafter, in a case where the human sensor 302 has detected that the measurement target person has entered the toilet room 300, the computation section 22 included in the CPU 1 carries out a urination amount information storing process described later, thereby measuring the urination amount. Specifically, the computation section 22 integrates urination amounts measured during a predetermined time period (e.g., one day) or the urination amount over a predetermined number of tunes of urination (e.g., one time). The information storage section 24 included in the CPU 1 generates a record (see FIG. 8) including information on the urination amount measured by the computation section 22, and stores the record in the storage section 4 (S3).

The comparison section 25 included in the CPU 1 reads information on the body weight of the measurement target person from the storage section 4 (S4). In addition, the comparison section 25 included in the CPU 1 refers to a table (TB) (see FIG. 2) stored in the storage section 4 in which table the body weight and a threshold value are associated with each other, and selects a threshold value corresponding to the body weight of the measurement target person as described above (S5).

The comparison section 25 included in the CPU 1 compares the urination amount of the measurement target person with the threshold value corresponding to the body weight of the measurement target person (S6). Then, the evaluation section 27 of the CPU 1 determines the health status of the measurement target person on the basis of whether or not the body weight of the measurement target person exceeds the threshold value (S7). Specifically, in a case where the body weight of the measurement target person exceeds the threshold value, the evaluation section 27 included in the CPU 1 determines that the health status of the measurement target person is good (i.e., normal). On the other hand, in a case where the body weight of the measurement target person is not more than the threshold value, the evaluation section 27 included in the CPU 1 determines that the health status of the measurement target person may be becoming bad.

Thereafter, the evaluation result processing section 28 included in the CPU 1 outputs the result of determining the health status of the measurement target person to the output section 9 and/or the external device. For example, in a case where the evaluation section 27 has determined that the health status of the measurement target person is good, the evaluation result processing section 28 included in the CPU 1 outputs, for example, the message “You are in good health condition” on the display of the output section 9. On the other hand, in a case where the evaluation section 27 has determined that the health status of the measurement target person may be becoming bad, the evaluation result processing section 28 included in the CPU 1 outputs, for example, the message “The amount of your urine has been decreasing. Please be careful of your health condition” on the display of the output section 9. In a case where the comparison section 25 uses a threshold value to determine whether the measurement target person has a particular disease, then such a message for that disease as “You may have urinary calculus or renal failure” may be outputted on the display of the output section 9. The health status determining process ends there.

The above example uses a threshold value corresponding to the body weight of a measurement target person. The health status determining process may use the same threshold value (i.e., predetermined value) regardless of the body weight of a measurement target person. In this case, (i) the threshold value is a normal value of the urination amount with respect to the body weight of the measurement target person (e.g., an upper limit value or lower limit value of a normal range), and (ii) in S6, the comparison section 25 compares the threshold value with the urination amount divided by the body weight of the measurement target person (i.e., the urination amount of a measurement target person with respect to the body weight of the measurement target person).

### Flow of Urination Amount Information Storing Process

FIG. 7 is a flowchart illustrating the flow of a urination amount information storing process carried out by the CPU 1 included in the urination amount measurement device 100. The memory 2 stores a program corresponding to the flowchart. The CPU 1 reads the program from the memory 2 and executes the program.

As shown in FIG. 7, in a case where the urination amount measurement device 100 has been turned on, the CPU 1 starts the program stored in the memory 2 and enters an IDLE state (i.e., wait state) (S11). In the IDLE state, the computation section 22 included in the CPU 1 communicates with the sensor 31 and the human sensor 302 via the communication section 13. In a case where the sensor 31 has detected that the water level of the collected water 36 has risen, or in a case where the human sensor 302 has detected a measurement target person entering the toilet room 300, the computation section 22 included in the CPU 1 determines that the measurement target person has started urination (S13).

If the water level of the collected water 36 detected by the sensor 31 exceeds a predetermined threshold value (i.e., YES in S13), the computation section 22 included in the CPU 1 uses the timer 3 to time the urination (S15). Every time the computation section 22 of the CPU 1 receives from the timer 3 a notification that a unit time has elapsed (S17), the computation section 22 of the CPU 1 calculates the urination amount of the measurement target person in the unit time (S19). The computation section 22 included in the CPU 1 stores the calculated data on the urination amount in the memory 2 (S21). Until the urination ends, the computation section 22 included in the CPU 1 calculates the urination amount of the measurement target person for each unit time, and integrates the urination amounts of the measurement target person calculated for each unit time.

In a case where the water level of the collected water 36 has not been increased for a predetermined period (YES in S23), the computation section 22 included in the CPU 1 determines that the urination of the measurement target person has ended, and stops the timer 3 (S25). Alternatively, in a case where the human sensor 302 has detected the measurement target person leaving the toilet room 300, the computation section 22 included in the CPU 1 may determine that the urination of the measurement target person has ended. Alternatively, in a case where the sensor 31 has detected a change in the water level as a result of the collected water 36 being discharged, the computation section 22 included in the CPU 1 may determine that the urination of the measurement target person has ended.

In a case where the computation section 22 has determined that the urination of the measurement target person has ended, the information storage section 24 included in the CPU 1 generates a record (see FIG. 8) described above with use of data on the urination amount stored in the memory 2, and stores the generated record in the DB 10 (S27). Thereafter, the CPU 1 shifts to the IDLE state (S29). The urination amount information storing process ends there.

### Configuration of Database 10

FIG. 8 is a diagram schematically illustrating the configuration of a DB 10 stored in the storage section 4 included in the urination amount measurement device 100. As shown in FIG. 8, the DB 10 includes one or more records Ri (where i=1, 2, 3, . . . , n). Each record Ri includes date-and-time data D1, a status ST, and urination amount data D2.

The date-and-time data D1 indicates the date and time at which the sensor 31 detected urination of a measurement target person. Urination amount data D2 includes information on the amount of urination over a predetermined number of times or predetermined time period. A status ST indicates the health status of the measurement target person as determined on the basis of the body weight and urination amount of the measurement target person. For example, in a case where the CPU 1 has determined that the urination amount of the measurement target person is abnormal, that is, the health status of the measurement target person may be becoming bad, as a result of the health status determining process, the record Ri stored in the DB 10 indicates “abnormal” as the status ST. On the other hand, in a case where the urination amount of the measurement target person is normal as a result of the health status determining process, and the CPU 1 has determined that the health status of the measurement target person is good, the record Ri indicates “normal” as the status ST. Urination amount data D2 shows the amount of urination by the measurement target person per one time or one day.

The record Ri may further include information on the number of times the measurement target person has entered the toilet room 300 within a day or a predetermined time period, and (ii) information on the number of times the measurement target person has urinated within a day or a predetermined time period.

In the example described above, the body weight of a measurement target person is measured in advance and stored in the storage section 4. The means and time point for measuring the body weight of a measurement target person are not limited to the above. The body weight of the measurement target person may be measured during measurement of the urination amount with use of a measuring instrument such as a body weight meter included in the toilet tool 30. Alternatively, the CPU 1 may receive, via the communication section 13, data on the body weight of a measurement target person from log information recorded in an external device or the like such as a body weight meter. Alternatively, an image recognition section such as a camera provided to the toilet tool 30 may identify the individual and estimate the body weight of the individual.

### Embodiment 2

For Embodiment 1, the urination amount information storing process shown in FIG. 7 starts when the urination amount measurement device 100 is turned on. For the present embodiment, in a case where the number of times a measurement target person has entered the toilet room 300 or the number of times the measurement target person has urinated into the toilet tool 30 exceeds a predetermined value, the CPU 1 starts a urination amount information storing process.

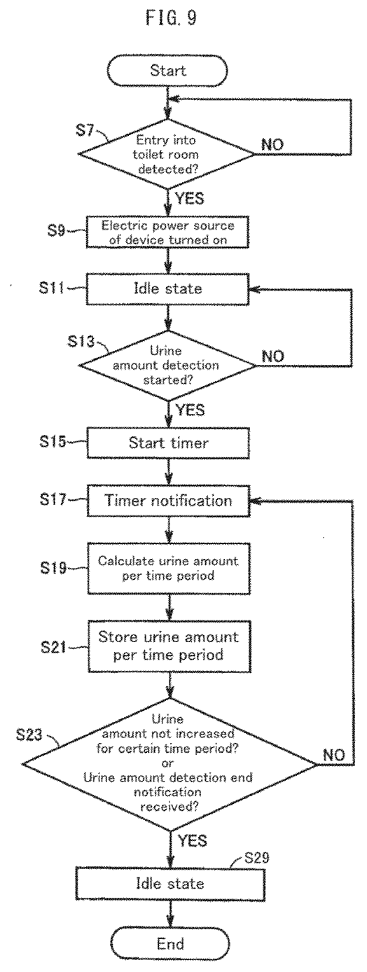

FIG. 9 is a flowchart illustrating the flow of a urination amount information storing process according to the present embodiment. FIG. 9 is a flowchart obtained by adding steps S7 and S9 to the flowchart shown in FIG. 7. The following descriptions of the present embodiment do not include a description of the steps other than steps S7 and S9.

### Flow of Urination Amount Information Storing Process

As shown in FIG. 9, in the urination amount information storing process for the present embodiment, the CPU 1 determines whether or not it has received a detection signal from the human sensor 302, whereby the CPU 1 determines whether or not a measurement target person has entered the toilet room 300 (S7). In a case where a measurement target person has entered the toilet room 300 (i.e., YES in S7), the CPU 1 turns on the electric power source 5 (see FIG. 2) included in the urination amount measurement device 100 (S9). This allows the electric power source 5 included in the urination amount measurement device 100 to provide electric power to the individual sections included in the urination amount measurement device 100. In a case where the human sensor 302 has detected that the measurement target person has exited the toilet room 300 in the middle of the urination amount information storing process according to the present embodiment, the CPU 1 executes an interrupt process and changes the electric power source 5 of the urination amount measurement device 100 from the on state to the off state, thereby stopping electric power from being provided to the individual sections of the urination amount measurement device 100. Prior to the CPU 1 turning the electric power source 5 off, the CPU 1 may use data on the urination amount stored in the memory 2 to generate a record (see FIG. 8) and store the generated record in the DB 10.

### Embodiment 3

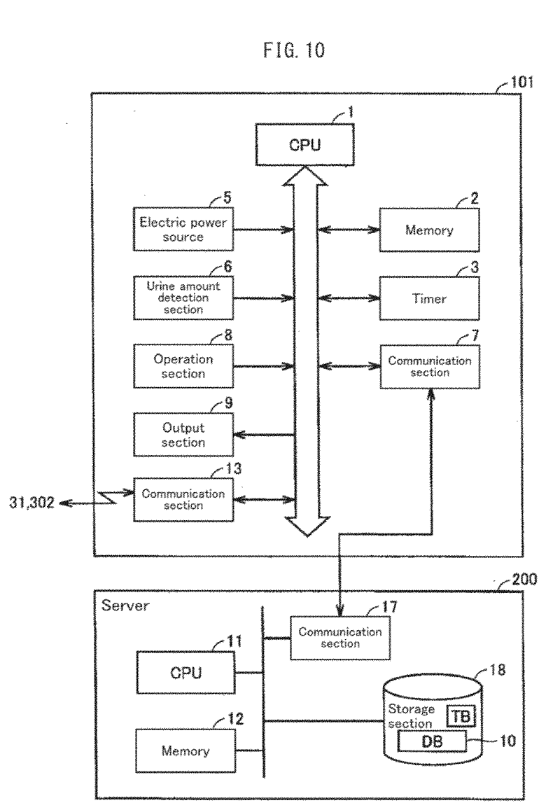

For Embodiments 1 and 2, the urination amount measurement device 100 includes a storage section 4 with a DB 10 stored therein. For the present embodiment, the storage section 4 is included in the server 200 as an external information processing device. FIG. 10 is a diagram schematically illustrating the configuration of a urination amount measurement device 101 (i.e., urine information processing device) according to the present embodiment.

The configuration of the urination amount measurement device 101 shown in FIG. 10 is compared with the configuration of the urination amount measurement device 100 of the Embodiment 1 as follows. That is, the urination amount measurement device 101 does not include the storage section 4. The urination amount measurement device 101 instead includes a communication section 7 for communicating with the server 200. The configuration of the urination amount measurement device 101 is otherwise similar to that of the urination amount measurement device 100 according to Embodiment 1.

A storage section 18 included in the server 200 stores a DB 10 including a record Ri of the urination amount (see FIG. 8). The urination amount measurement device 101 transmits a record Ri generated as a result of the urination amount information storing process shown in FIG. 7 or 9 to the server 200 via the communication section 7. The CPU 11 included in the server 200 stores the record Ri, received by the communication section 17, in the DB 10 included in the storage section 18. Instead of the urination amount measurement device 101, the CPU 11 included in the server 200 may generate a record Ri on the basis of data as a result of the detection by the sensor 31. In this case, the CPU 11 determines the health status of the measurement target person with use of data as a result of the detection by the sensor 31, generates a record Ri including the result of the determination, and stores the record Ri in the DB 10.

The CPU 11 included in the server 200 transmits data indicating the determination result to the urination amount measurement device 101 via the communication section 35. The urination amount measurement device 101 outputs the data received from the server 200 to the output section 9.

The above configuration eliminates the need for the urination amount measurement device 101 to make a health status determination. The above configuration allows the urination amount measurement device 101 to output data provided by the server 200, i.e., the determination result of the health status. The measurement target person can check their own health status by referring to data outputted from the output section 9.

### Embodiment 4

For the present embodiment, step S27 of the urination amount information storing process (see FIG. 7) is a step in which the CPU 1 selectively stores some records Ri in the DB 10 instead of storing all records Ri in the DB 10. Specifically, in a case where the user has inputted, to the urination amount measurement device 100 or 101 via the operation section 8, an instruction to not store a record Ri, the CPU 1 does not store a record Ri in the DB 10. According to the above configuration, in a case where the user or the measurement target person has inputted via the operation section 8 an instruction to not register a record Ri, the CPU 1 does not register a record Ri including information on a particular urination amount in the DB 10.

The human sensor 302 does not use infra-red radiation to detect a measurement target person entering the toilet room 300 or exiting the toilet room 300. Instead, the human sensor 302 detects the intensity of the electric field of a weak radio signal outputted by the urination amount measurement device 100 or 101. The human sensor 302 may, with the above configuration, detect a measurement target person entering the toilet room 300 or exiting the toilet room 300.

### Embodiment 5

In one embodiment, the CPU 1 may determine the health status of a measurement target on the basis of the number of times of urination over a predetermined time period and the time necessary for urination. In this case, the condition under which the CPU 1 determines the health status of a measurement target person may be, for example, the following condition: The urine amount with respect to the body weight of a measurement target person (e.g., urine amount of a measurement target person per time or per day) exceeds a threshold value, and the number of times of urination by the measurement target person over a predetermined time period (e.g., per day) exceeds a threshold value. The determination condition may be as follows: The urine amount with respect to the body weight of a measurement target person (e.g., the urine amount of a measurement target person per time or per day) exceeds a threshold value, and the time required for a measurement target person to urinate (e.g., the average time required for urination for a day) exceeds a threshold value. Further, the determination condition may be a condition based on a combination of the urination amount of a measurement target person, the number of times of urination of the measurement target person, and the time required for urination by the measurement target person. What information to combine and how to set a threshold value may be selected according to the health status to be determined by the CPU 1. For example, in a case where it is known that if a person suffers from a particular disease, the person's urine amount per day with respect to the body weight increases, and the person's number of times of urination per day also increases, the determination condition includes the urine amount of a measurement target person per day and the number of times of urination. by the measurement target person per day. Alternatively, the CPU may determine the health status of a measurement target on the basis of at least either the number of times of urination or the time required for urination. The time necessary for urination may be a time necessary per time or the sum of the time necessary over a predetermined time period (e.g., one day).

The number of times the measurement target person has urinated over a predetermined time period can be determined by, with use of the human sensor 302, counting the number of times the measurement target person has entered the toilet room 300 and the number of times the measurement target person has exited the toilet room 300. The amount of time a measurement target person takes for urination can be measured with use of the timer 3. The CPU 1 may use, as a time point at which a measurement target person started urination, (i) a time point at which the human sensor 302 has detected the measurement target person entering the toilet room 300 or (ii) a time point at which the sensor 31 has detected an increase in the water level. Similarly, the CPU 1 may use, as a time point at which a measurement target person ended urination, (i) a time point at which the human sensor 302 has detected the measurement target person exiting the toilet room 300, (ii) a time point at which the sensor 31 has detected that the water level has been increased, and (iii) a time point at which the toilet tool 30 has been flushed.

### Embodiment 6

Embodiments 1 to 5 are each a case where the measurement target is a human being. For the present embodiment, the measurement target is a cat.

### System Overview

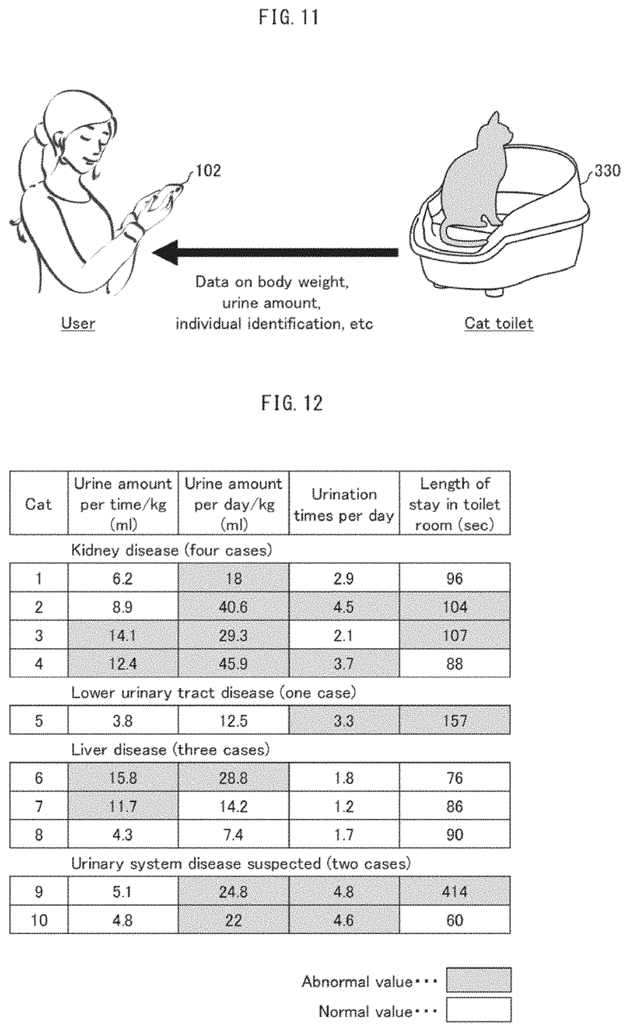

FIG. 11 is a diagram schematically illustrating a urine information processing system according to the present embodiment. As shown in FIG. 11, the urine information processing system according to the present embodiment includes a urination amount measurement device 102 (urine information processing device) and a cat toilet 330 (toilet tool). The configuration of the urination amount measurement device 102 according to the present embodiment is identical to that of the urination amount measurement device 100 described above as Embodiment 1 (see FIGS. 1 and 2). For the present embodiment, the process carried out by the computation section 22 included in the CPU 1 differs from those for Embodiments 1 to 5. The cat toilet 330 includes a sensor 331 (i.e., urination detection section, measurement target detection section) at a bottom portion thereof. Specifically, the sensor 331 is a weight sensor. The urine information processing system according to the present embodiment does riot include the sensor 31 and the human sensor 302 included in the urine information processing system according to Embodiment 1, but instead includes the sensor 331.

The sensor 331 is installed at a bottom portion of the cat toilet 330. As shown in FIG. 11, in a case where a cat as a measurement target moves onto the cat toilet 330, the sensor 331 detects the weight of the cat. Thereafter, the sensor 331 transmits data on the detected weight of the cat to the urination amount measurement device 102 of the user (i.e., the owner of the cat) by radio communication.

In a case where the CPU 1 has received data on the weight of the cat from the sensor 331, the CPU 1 (see FIG. 2) included in the urination amount measurement device 102 determines that the cat is about to start excreting, and exits the IDLE state. Further, the CPU 1 receives data for individual identification of the cat from a chip provided on a collar or the like of the cat on the cat toilet 330 (not shown). The data for individual identification of the cat is used by the information storage section 24 (see FIG. 1) of the CPU 1 to associate a record as described for Embodiment 1 with (ii) information on the identified cat. The computation section 22 (see FIG. 1) of the CPU 1 outputs data on the weight of the cat received from the sensor 331 to the comparison section 25 included in the CPU 1. In a manner similar to the above embodiments, the comparison section 25 selects a threshold value corresponding to the body weight of the cat as a measurement target.

In a case where the cat has moved off the cat toilet 330 after the cat has urinated on the cat toilet, the sensor 331 detects the weight of urine excreted by the cat. The sensor 331 transmits data on the detected weight to the urination amount measurement device 102. The computation section 22 measures the urination amount with use of the data on the detected urine weight. In other words, the computation section 22 converts data on the urine weight into data on the urination amount. The computation section 22 according to the present embodiment outputs data on the urination amount measured by the computation section 22 in this manner to the information storage section 24 and comparison section of the CPU 1. The processes carried out by the individual sections other than the computation section 22 included in the CPU 1 are as described for Embodiment 1. For the present embodiment, descriptions thereof will be omitted. In a case where the CPU 1 uses the measurement target urine weight as a threshold value to determine the health status of the measurement target, the conversion described above can be omitted.

### Software Implementation Example

Control blocks of urination amount measurement device 100, 101, and 102 (particularly, the computation section 22, the determination unit 120, and the output control unit 130) can be realized by a logic circuit (hardware) provided in an integrated circuit (IC chip) or the like.

### Reference

FIG. 12 shows exemplary data obtained as a result of the Applicant's investigation into the correlation between (i) the urination amount and the like of a measurement target and (ii) particular diseases. FIG. 12 shows data for a case where the measurement target is a cat. The data shown in FIG. 12 includes (i) the urination amount per time (i.e., the value of the urination amount divided by the body weight of the measurement target), (ii) the urination amount per day (i.e., the value of the urination amount divided by the body weight of the measurement target), (iii) the number of times the measurement target has urinated per day, and (iv) the time period during which the measurement target stayed on the cat toilet 330. In the data shown in FIG. 12, any cell with an abnormal value is shaded. As a result of investigating healthy measurement targets, the Applicant has discovered that the upper limit of a normal value is as follows: the urination amount per time: 10 ml/kg, the urination amount per day: 15 ml/kg, the number of times of urination per day: 3.0 times per day, the length of stay: 100 sec. The table in FIG. 12 shows, as an abnormal value, any value above the upper limit of a normal value discovered by the Applicant. Although not shown in FIG. 12, any value less than the lower limit of a normal value may also be treated as an abnormal value.

Thus, the urination amount and the like of a measurement target correlate with the health status of the measurement target. This allows the urination amount measurement device 100, 101, 102 to determine the health status of a measurement target on the basis of information such as the urination amount of the measurement target. Which of the urination amount of a measurement target, the urination count of a measurement target, and the length of stay of a measurement target has an abnormal value differs from disease to disease. Appropriate combinations of these allow the urination amount measurement device 100, 101, 102 to determine what disease the measurement target is suffering from (i.e., what disease the measurement target is likely to be suffering from).

### Recap

A urine information processing device (urination amount measurement device 100, 101, 102) in correspondence with a first aspect of the present invention is a urine information processing device including: a urination amount measurement section (computation section 22) configured to measure an amount of urination by a measurement target that uses a toilet tool; a comparison section (25) configured to compare (i) the amount of the urination by the measurement target, the amount having been measured by the urination amount measurement section, with (ii) a predetermined value corresponding to a body weight of the measurement target or compare (i) a quotient of the amount of the urination by the measurement target, the amount having been measured by the urination amount measurement section, divided by the body weight of the measurement target with (ii) the predetermined value; a health status determination section (evaluation section 27) configured to determine a health status of the measurement target on a basis of a result of the comparison by the comparison section; and a determination result presentation section (evaluation result processing section 28) configured to present a result of the determination by the health status determination section.

The above configuration allows the health status of a measurement target to be determined on the basis of the urination amount of the measurement target. In a case where the measurement target is a user (that is, a human being), the user can easily check their health status by referring to the result of the determination outputted by the determination result presentation section.

A urine information processing device in accordance with a second aspect of the present invention is arranged as in the first aspect and may be further configured such that the comparison section is configured to compare (i) quotient of the amount of the urination by the measurement target, the amount having been measured by the urination amount measurement section, divided by the body weight of the measurement target with (ii) a predetermined value. The above configuration allows the health status of a measurement target to be determined on the basis of the urination amount of the measurement target.

A urine information processing device in accordance with a third aspect of the present invention is arranged as in the first or second aspect and may be further configured such that the urination amount measurement section is configured to calculate an integrated value of a predetermined number of amounts of the urination by the measurement target, and the health status determination section is configured to determine the health status of the measurement target in a case where the integrated value of the predetermined number of the amounts of the urination by the measurement target has deviated from the predetermined value by not less than a certain value. The above configuration makes it possible to determine the health status of a measurement target correlated to the integrated value of a predetermined number of amounts of urination by the measurement target.

A urine information processing device in accordance with a fourth aspect of the present invention is arranged as in the first or second aspect and may be further configured such that the urination amount measurement section is configured to calculate an integrated value of amounts measured of the urination by the measurement target over a predetermined time period, and the health status determination section is configured to determine the health status of the measurement target in a case where the integrated value of the amounts measured of the urination by the measurement target over the predetermined time period has deviated from the predetermined value by not less than a certain value. The above configuration makes it possible to determine the health status of a measurement target correlated to the integrated value of amounts measured of urination by the measurement target over a predetermined time period.

A urine information processing device (urination amount measurement device 100, 101, 102) in correspondence with a fifth aspect of the present invention is a urine information processing device including: a urination information measurement section (computation section 22) configured to (i) sense a measurement target that uses a toilet tool (30) and (ii) measure at least either the number of times of urination by the measurement target over a predetermined time period or a time period necessary for the measurement target to urinate; a comparison section (25) configured to compare either the number of times of the urination by the measurement target, the number having been measured by the urination information measurement section, with a first predetermined value for the number of times of urination or the time period necessary for the measurement target to urinate, the time period having been measured by the urination information measurement section, with a first predetermined value for a time period necessary for urination; a health status determination section (evaluation section 27) configured to determine a health status of the measurement target on a basis of a result of the comparison by the comparison section; and a determination result presentation section (evaluation result processing section 28) configured to present a result of the determination by the health status determination section.

The above configuration allows the health status of a measurement target to be determined correlated to at least one of the number of times of urination by the measurement target and the time it takes for the measurement target to urinate. In a case where the measurement target is a user (that is, a human being), the user can easily check their health status by referring to the result of the determination outputted by the determination result presentation section.

The urine information processing device according to the foregoing aspects of the present invention may be realized by a computer. In this case, the present invention encompasses: a control program for the urine information processing device which program causes a computer to operate as each of the foregoing sections (software elements) of the urine information processing device so that the urine information processing device can be realized by the computer; and a computer-readable storage medium storing the control program therein.

The present invention is not limited to the embodiments, but can be altered by a skilled person in the art within the scope of the claims. The present invention also encompasses, in its technical scope, any embodiment derived combining technical means disclosed in differing embodiments. Further, it is possible to form a new technical feature by combining the technical means disclosed in the respective embodiments.

### REFERENCE SIGNS LIST

22 Computation section (urination amount measurement section, urination information measurement section)

25 Comparison section

27 Evaluation section (health status determination section)

28 Evaluation result processing section (determination result presentation section)

30 Toilet tool

31 Sensor (urination detection section)

100, 101, 102 Urination amount measurement device (urine information processing device)

302 Human sensor (measurement target detection section)

330 Cat toilet (toilet tool)

331 Sensor (urination detection section, measurement target detection section)

1. A urine information processing device, comprising:
a urination amount measurement section configured to measure an amount of urination by a measurement target that uses a toilet tool;
a comparison section configured to compare (i) the amount of the urination by the measurement target, the amount having been measured by the urination amount measurement section, with (ii) a predetermined value corresponding to a body weight of the measurement target and an average value of past urination amounts of the measurement target;
a health status determination section configured to determine a health status of the measurement target on a basis of a result of the comparison by the comparison section; and
a determination result presentation section configured to present a result of the determination by the health status determination section.

2. The urine information processing device according to claim 1, wherein
the comparison section is configured to compare (i) a quotient of the amount of the urination by the measurement target, the amount having been measured by the urination amount measurement section, divided by the body weight of the measurement target with (ii) a predetermined value.

3. The urine information processing device according to claim 1, wherein
the urination amount measurement section is configured to calculate an integrated value of a predetermined number of amounts of the urination by the measurement target, and
the health status determination section is configured to determine the health status of the measurement target in a case where the integrated value of the predetermined number of the amounts of the urination by the measurement target has deviated from the predetermined value by not less than a certain value.

4. The urine information processing device according to claim 1, wherein
the urination amount measurement section is configured to calculate an integrated value of amounts measured of the urination by the measurement target over a predetermined time period, and
the health status determination section is configured to determine the health status of the measurement target in a case where the integrated value of the amounts measured of the urination by the measurement target over the predetermined time period has deviated from the predetermined value by not less than a certain value.

5. A urine information processing device, comprising:
a urination information measurement section configured to (i) sense a measurement target that uses a toilet tool and (ii) measure either the number of times of urination by the measurement target over a predetermined time period or a time period necessary for the measurement target to urinate;
a comparison section configured to compare either the number of times of the urination by the measurement target, the number having been measured by the urination information measurement section, with a first predetermined value for the number of times of urination or the time period necessary for the measurement target to urinate, the time period having been measured by the urination information measurement section, with a first predetermined value for a time period necessary for urination;
a health status determination section configured to determine a health status of the measurement target on a basis of a result of the comparison by the comparison section; and
a determination result presentation section configured to present a result of the determination by the health status determination section.

6. A urine information processing system, comprising:
a urine information processing device according to claim 1; and
a urination detection section provided for the toilet tool and configured to sense the urination by the measurement target or a measurement target detection section configured to sense the measurement target that uses the toilet tool.

7. A non-transitory computer-readable storage medium storing therein a control program for causing a computer to function as a urine information processing device according to claim 1, the program causing the computer to function as each of the urination amount measurement section, the comparison section, the health status determination section, and the determination result presentation section.
